(19) United States
(12) Patent Application Publication
(10) Pub. No.: US 2024/0225709 A1
(43) Pub. Date: Jul. 11, 2024

(54) OPENING WEDGE TAP INSTRUMENT FOR PERFORMING OSTEOTOMY AND METHOD OF USING THEREOF

(71) Applicants: Khay Yong SAW, Kuala Lumpur (MY); Caroline Siew Yoke JEE, Kuala Lumpur (MY)

(72) Inventors: Khay Yong SAW, Kuala Lumpur (MY); Caroline Siew Yoke JEE, Kuala Lumpur (MY)

(21) Appl. No.: 18/333,245

(22) Filed: Jun. 12, 2023

(30) Foreign Application Priority Data
Jan. 9, 2023 (MY) PI2023000126

(51) Int. Cl. A61B 17/86; A61B 17/16; A61B 17/17

(52) U.S. Cl. CPC A61B 17/8635; A61B 17/1615; A61B 17/1764; A61B 17/157

(57) ABSTRACT

The disclosure provides an osteotomy tool, namely an opening wedge tap Instrument, for prying open an osteotomy cut to a pre-determined degree to correct a tibia deformity. The osteotomy tool includes a screw and a shank. The screw and shank have a through-hole aligned along the osteotomy tool's longitudinal axis to form a passage for receiving a guidewire, which helps to position and guide the osteotomy tool's movement. While the osteotomy tool moves into the osteotomy cut, the osteotomy tool pries open the ostomy cut. The disclosure also provides a method for performing an osteotomy using the osteotomy tool. The method includes performing an osteotomy cut on a tibia, mounting a guidewire into the osteotomy cut, inserting the guidewire into the opening wedge tap instrument's through-hole, screwing the opening wedge tap instrument into the osteotomy cut to pry open the osteotomy cut to a degree for correcting the tibia deformity.

### FIELD

The disclosure generally relates to an open wedge osteotomy. Specifically, the disclosure relates to an osteotomy tool, namely an opening wedge tap instrument, for pry opening an osteotomy cut and a method of using this opening wedge tap instrument to perform an open wedge osteotomy.

### BACKGROUND

The open wedge osteotomy generally is a surgical operation to realign the tibia. Typically, the open wedge osteotomy involves performing a cut on the tibia, opening the cut to a pre-determined degree to realign the tibia, and then fixating the realigned tibia. The open wedge osteotomy can treat or fix abnormalities or conditions of the knee. There are different types of open wedge osteotomy. In some examples, the open wedge osteotomy can be categorized based on where the cut is performed. A high tibial osteotomy (HTO) involves cutting the tibia above the tuberosity of the tibia. An osteotomy below the tuberosity involves cutting the tibia below the tuberosity.

### SUMMARY

The embodiments herein provide an opening wedge tap instrument for pry opening an osteotomy cut and a method of using the opening wedge tap instrument to perform an open wedge osteotomy.

In an embodiment, the opening wedge tap instrument includes an elongated body that includes a shank and a screw. The screw is configured to a frustoconical shape. The screw includes a thread having a flank, crest, and root. The flank includes a driving side and backside, and the driving side is not symmetrically related to the backside. The driving side has a smaller gradient or smaller slope than the backside.

In an embodiment, the driving side includes two edges having an angle in a range of larger than 120° to less than 180°.

In an embodiment, the crest includes a flat edge or surface.

In an embodiment, the root includes a flat edge or surface.

In an embodiment, the thread has a depth of at or about 0.5 mm. In an embodiment, the thread has a pitch of at or about 2.5 mm.

In an embodiment, the screw has a cone angle in a range of at or about 8° to at or about 10°. In an embodiment, the screw has a minimum diameter of at or about 3.91 mm and a maximum diameter of at or about 10.03 mm. In an embodiment, the screw has a length of at or about 35 mm.

In an embodiment, the screw is made of biocomposite or bioceramic material capable of promoting bone growth.

In an embodiment, the shank is removably attached to the screw. In an embodiment, the shank is irremovably attached to the screw.

In an embodiment, the shank has a diameter or thickness of at or about 4 mm to at or about 8 mm. In an embodiment, the shank has a length of at or about 65 mm.

In an embodiment, the opening wedge tap instrument has a through-hole extending along the longitudinal axis thereof to receive a guiding rod or wire.

In an embodiment, the opening wedge tap instrument further includes a handle. In an embodiment, the elongated body is irremovably or removably attached to the middle of the handle, and the handle includes an elongated body or knob.

In an embodiment, the method includes performing an osteotomy cut on a tibia, mounting a guidewire into the osteotomy cut, inserting the guidewire into the through-hole of the opening wedge tap instrument, and screwing the opening wedge tap instrument into the osteotomy cut to pry open the osteotomy cut a degree for correcting deformity of the tibia.

In an embodiment, the method further includes detaching the shank from the screw and fixating the osteotomy cut with a fixation plate. Alternatively, the method further includes fixating the pry-opened osteotomy cut with a fixation plate and unscrewing the screw out of the tibia.

### BRIEF DESCRIPTION OF THE DRAWINGS

References are made to the accompanying drawings that form a part of this disclosure, and which illustrate embodiments in which the device and method described in this specification can be practiced.

FIG. 1 schematically illustrates an opening wedge tap instrument according to an embodiment.

FIG. 2 schematically illustrates the 2-2 section of the opening wedge tap instrument of FIG. 1, according to an embodiment.

FIG. 3A schematically illustrates an enlarged view of screw threads according to an embodiment.

FIG. 3B schematically illustrates an enlarged view of screw threads according to another embodiment.

FIG. 3C schematically illustrates an enlarged view of screw threads according to another embodiment.

FIG. 3D schematically illustrates an enlarged view of screw threads according to another embodiment.

FIG. 4 schematically illustrates an opening wedge tap instrument, according to another embodiment.

Like reference numbers represent like parts throughout different views and embodiments.

### DETAILED DESCRIPTION

The disclosure provides an opening wedge tap instrument to pry open an osteotomy cut in an open wedge osteotomy. The disclosure also provides a method to perform an open wedge osteotomy using the opening wedge tap instrument.

The term “a,” “an,” or “the” covers both the singular and the plural reference unless the context dictates otherwise. The terms “comprising,” “having,” “including,” and “containing” are open-ended terms, which means “including but not limited to,” unless otherwise indicated.

Certain values herein are preceded by the term “about.” The term “about” herein provides literal support for the exact value that it precedes, as well as a range that is near to or approximately the value that the term precedes. In an embodiment, the range is from 70% to 130% of the exact value that the term “about” precedes. In an embodiment, the range is from 80% to 120% of the exact value that the term “about” precedes. In an embodiment, the range is from 90% to 110% of the exact value that the term “about” precedes. In an embodiment, the range is from 99% to 101% of the exact value that the term “about” precedes. For example, if the exact value is 100, the range from 70% to 130% of the exact value is 70 to 130.

The open wedge osteotomy relies on surgical tools. In an open wedge osteotomy, a surgeon uses an oscillating saw or osteotome to make an osteotomy cut at a selected position on a tibia, after calculating the correction degree. In an embodiment, the osteotomy cut can be made via multiple drilling using an osteotomy guide device. The osteotomy guide device is configured to ensure the multiple drillings occur around the tibia and across a horizontal cross-sectional plane of the tibia so that directions of the drillings are substantially parallel to or on the same horizontal cross-sectional plane of the tibia. The surgeon then uses a chisel to open the osteotomy cut. Afterward, the surgeon uses a spreader to pry open the osteotomy cut to a degree for correcting the tibia's alignment.

This opening wedge tap instrument disclosed herein can replace the chisel and the spreader. The opening wedge tap instrument can pry open the osteotomy cut to the desired degree for correcting, for example, tibia deformity, as it gradually advances into the cut in a screw-in motion. In an embodiment, one opening wedge tap instrument is used to pry open the osteotomy cut. In an embodiment, two or more opening wedge tap instruments are used to collaboratively and cooperatively pry open the osteotomy cut. The two or more opening wedge tap instruments can be coordinately screwed into different locations of the osteotomy cut. In an embodiment, an osteotomy cut can be opened via multiple drillings with the opening wedge tap instrument.

The opening wedge tap instrument exhibits significant advantages over the chisel and spreader in opening the osteotomy cut for correcting the tibia deformity. The advantages will become readily apparent from the following description which should be read in conjunction with the accompanying drawings.

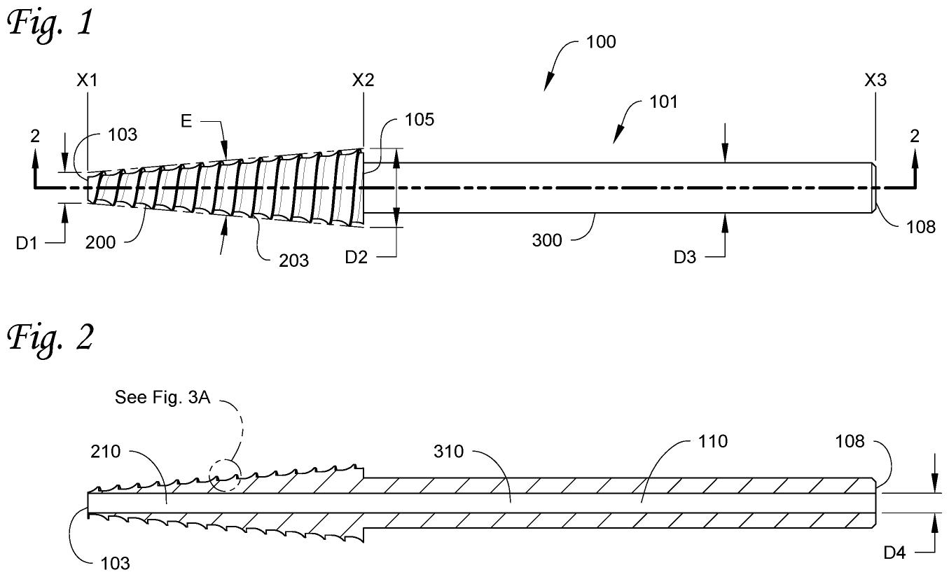

Referring to FIG. 1, FIG. 1 illustrates an opening wedge tap instrument 100 according to an embodiment. This embodiment can include an elongated body 101 having an end 103 and an end 108 opposing the end 103. The body 101 can include a screw 200 and a shank 300 in order from the end 103 to the end 108.

The screw 200 is generally frustoconically shaped. In an embodiment, the frustoconical shape of the screw 200 can have a cone angle, indicated by E. The cone angle E can fall within a range from about 2° to about 30°. In an embodiment, the lower limit of the range can be 3°, 4°, 5°, 6°, 7°, 8°, 9°, 10°, 11°, or 12°. In an embodiment, the upper limit of the range can be 20°, 21°, 22°, 23°, 24°, 25°, 26°, 27°, 28°, or 29°. In an embodiment, the cone angle E is at or about 2°. In an embodiment, the cone angle E is at or about 10°. In an embodiment, the cone angle E is at or about 15°. In an embodiment, the cone angle E is at or about 30°. The screw 200 can include a thread 203 capable of converting rotational force to linear movement and prying open an osteotomy cut. The thread 203 has a gradually increasing diameter from the minimum at the end 103 to the maximum at the end 105. As the thread 203 moves into the osteotomy cut, the osteotomy cut would be gradually pried opened from a low degree to the desired correction degree, resulting in a more controllable realigning process for correcting deformed tibia as compared with chisel and spreader. D1 and D2 indicate the diameters of the end 103 and 105, respectively. In an embodiment, D1 is at or about 3.91 mm. In an embodiment, D2 is at or about 10.03 mm. In an embodiment, D1 is in a range of 2 mm to 10 mm.

In an embodiment, the thread 203 extends from the end 103 to the end 105. The thread 203 or screw 200 can have a length sufficient and suitable for prying opening the osteotomy cut as it gradually advances into the cut via its screw-in motion. This length can be indicated by, e.g., the distance between lines X1 and X2. In an embodiment, the thread 203 or screw 200 has a length of at or about 35 mm. In an embodiment, the thread 203 is right-handed. In an embodiment, the thread 203 is left-handed.

The end 103 can be configured to various styles or shapes, including but not limited to, for example, cone, oval, plain cup, flat, soft-tipped, and knurled cup, or the like, or a combination thereof. The end 103 can also be a blunt end or sharp end. The sharp end can include a sharp edge useful for drilling into the osteotomy cut. The blunt end can include a blunt edge. The blunt edge can include but is not limited to, for example, a flat edge, rounded edge, or the like, or a combination thereof.

The shank 300 can have a regular or irregular shape. In an embodiment, the shank 300 has a rod shape. In an embodiment, the shank 300 is a cylindrical rod, triangular rod, square rod, rectangular rod, pentagonal rod, hexagonal rod, octagonal rod, or the like, or a combination thereof.

The shank 300 can have a length suitable for a surgeon to drive the screw 200 into an osteotomy cut. The length is the most extended dimension, e.g., defined by the distance between lines X2 and X3. In an embodiment, the length can be in a range of at or about 20 mm to at or about 150 mm. In an embodiment, the upper limit of the range of the length can be at or about 100 mm, at or about 80 mm, or at or about 70 mm. In an embodiment, the lower limit of the range of the length can be at or about 25 mm, at or about 30 mm, or at or about 35 mm. In an embodiment, the shank 300 has a length of at or about 65 mm.

The shank 300 can have a diameter or thickness indicated by D3, e.g., the dimension extends perpendicular to the length direction, in a range of at or about 2 mm to at or about 30 mm. In an embodiment, the upper limit of the range of the diameter or thickness is at or about 10 mm, at or about 8 mm, or at or about 7 mm. In an embodiment, the lower limit of the range of the diameter or thickness is at or about 3 mm, at or about 5 mm. In an embodiment, the diameter or thickness is at or about 6.35 mm.

The shank 300 can be removably or irremovably attached to the screw 200. In an embodiment, the shank 300 is not detachable from the screw 200. In an embodiment, the shank 300 and the screw 200 are unitary one piece.

In an embodiment, the shank 300 is detachable from the screw 200. After the screw 200 pries open the osteotomy cut to the desired degree of correction via its screw-in motion, the shank 300 can be detached from the screw 200, and the screw 200 remains where it is to secure the osteotomy cut to the desired degree of correction to facilitate further operation. The ability of the shank 300 to detach from the screw 200 also allows the surgeon to select different screws with different sizes and cone angle α suitable for different patients. The shank 300 can attach to the screw 200 via a suitable mechanism, for example, male-female thread pairs.

Referring to FIG. 2, FIG. 2 schematically illustrates the 2-2 section of the opening wedge tap instrument 100 of FIG. 1, according to an embodiment. The 2-2 section is along the length direction of the instrument 100, e.g., the x-axis of the paper plane of FIG. 1. The screw 200 and the shank 300 have a hollow 210 and 310, respectively. The hollow 210 and 310 are aligned together along the length direction of the opening wedge tap instrument 100 to form a continuous through-hole 110 extending from the end 103 to the end 108. That is, the opening wedge tap instrument 100 is cannulated. In an embodiment, the through-hole 110 is configured to receive a guiding wire or rod, for example, k-wire, to facilitate the proper positioning and direct the proper screw-in motion of the opening wedge tap instrument 100. In an embodiment, the guiding rod or wire can ensure the opening wedge tap instrument adopts a position perpendicular to the tibia during the screw-in motion. In an embodiment, the through-hole can be used to deliver medication.

The diameters of the hollow 210 and 310 can be the same or different. In an embodiment, the hollow 210 and 310 can have the same diameter indicated by D4. In an embodiment, the diameters of the hollow 210 and 310 can be in a range of at or about 1 mm to at or about 5 mm. In an embodiment, the diameter of the hollow 210 and 310 is at or about 2.5 mm.

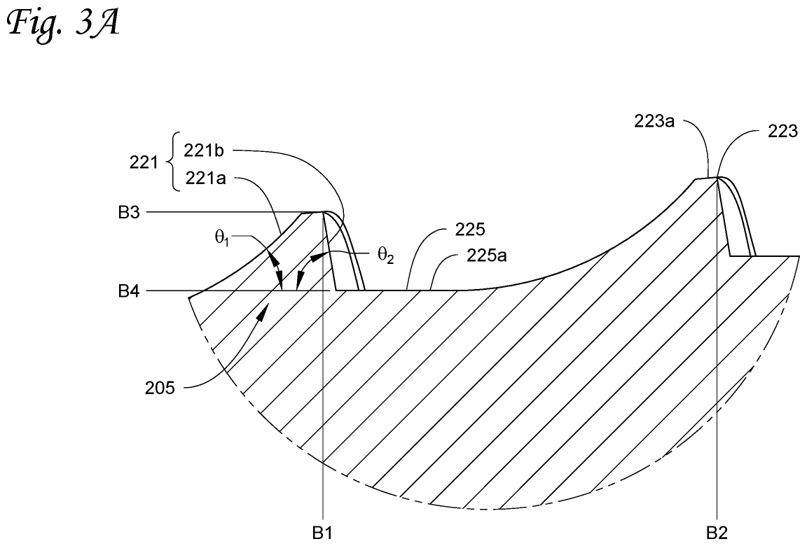

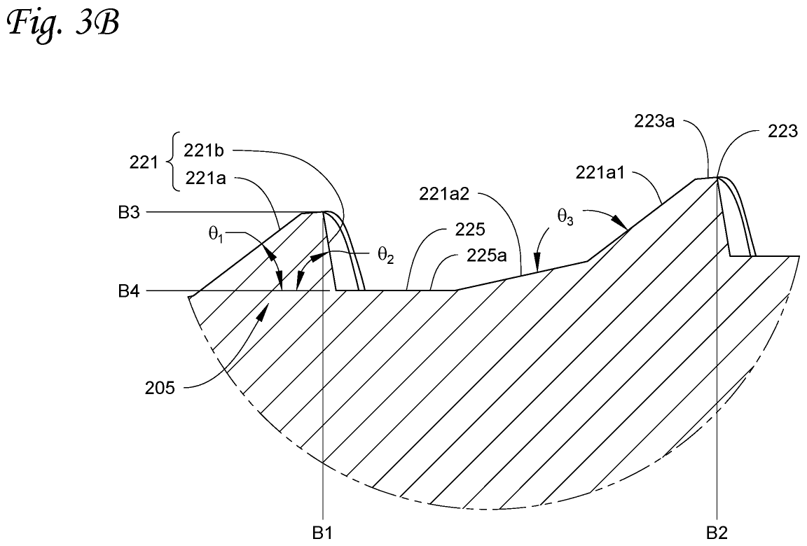

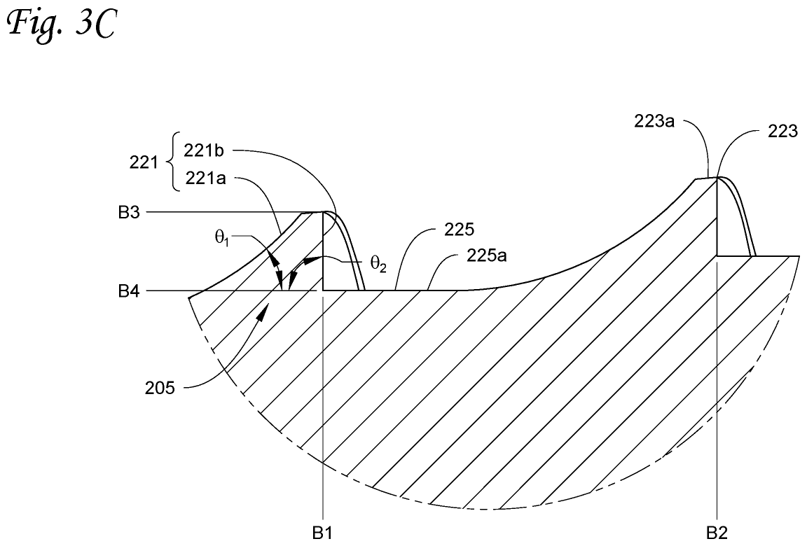

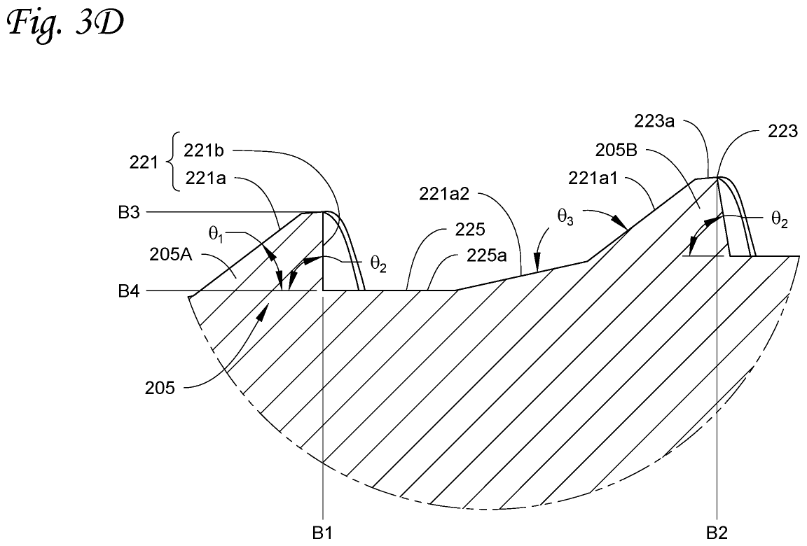

Referring to FIG. 3A, FIG. 3A is an enlarged view of screw threads according to an embodiment. FIG. 3A illustrates the shape and structure of the thread 203 according to an embodiment. The thread 203 includes a screw thread 205. The screw thread 205 can include a flank 221, a crest 223, and a root 225.

The flank 221 is the portion of the surface on either side of the screw thread 205. The flank 221 includes a driving side 221a, which is the leading edge of the screw thread 205, and a backside 221b, which is the trailing edge of the screw thread 205. In an embodiment, both the driving side 221a and the backside 221b make an oblique angle to the longitudinal axis of the screw 200, forming a trapezoidal-like thread form. The driving side 221a and the backside 221b can adopt angles θ₁ and θ₂, respectively, which are angles relative to the longitudinal axis of the screw 200. In an embodiment, the θ₁ and θ₂ can be in a range of large than 0° and less than 90°. In an embodiment, the θ₁ is smaller than the θ₂, and thereby the driving side can generally have a lower gradient or smaller slope than the backside. In an embodiment, the θ₂ is in a range of 30° to 60°. In an embodiment, the θ₁ is at or about 45°. In an embodiment, the θ₂ is in a range of 50° to 90°. In an embodiment, the θ₂ is at or about 90°. In an embodiment, the θ₂ is at or about 60°.

The flank 221 can be non-symmetric. For example, the driving side 221a is not a mirror image of the backside 221b. In an embodiment, the driving side 221a can be a smoothly extended edge. In an embodiment, as shown in FIG. 3B, the driving side 221a includes two edges 221a1 and 221a2. The two edges 221a1 and 221a2 can have an angle θ₃ in a range of larger than 120° to less than 180°. In an embodiment, the angle θ₃ is at or about 130° to at or about 165°.

The crest 223 is the peak edge of the screw thread 205, connecting the driving side 221a and the backside 221b. The crest 223 can include a flat surface, a curved surface, a sharp edge, or a combination thereof. In an embodiment, the crest 223 includes an edge or surface 223a. In an embodiment, the edge 223a is a flat edge. In an embodiment, the crest 223 includes a flat edge and a curved edge. In an embodiment, the crest 223 includes a flat edge and a sharp edge.

The root 225 is the bottom edge of the screw thread 205, connecting the adjacent flank 121 at the bottom of the screw thread 205. The root 225 includes a surface 225a. The surface 225a can be a flat edge. In an embodiment, the root 225 is a flat edge.

The screw thread 205 can have a pitch, indicated by the distance between lines B1 and B2, in a range of at or about 1 mm to at or about 4 mm. In an embodiment, the screw thread 205 can have a pitch of at or about 2.5 mm.

The screw thread 205 can have a depth, indicated by the distance between lines B3 and B4, in a range of at or about 0.1 mm to at or about 2 mm. In an embodiment, the screw thread 205 can have a depth of at or about 0.5 mm.

The screw thread 205's configuration, including the small slope of the driving side 221a, the flat edge of the crest, and the large slope of the backside 221b, are useful for prying open the osteotomy cut.

Referring to FIG. 3C, FIG. 3C is an enlarged view of screw threads according to another embodiment. The backside 221b of the screw thread 205 adopts a slope angle θ₂ of 90°.

Referring to FIG. 3D, FIG. 3D is an enlarged view of screw threads according to still another embodiment. The thread 203 can include different threads, for examples screw thread 205A and 205B. In an embodiment, the screw thread 205A and 205B can differ in their backside 221b. In an embodiment, the screw thread 205A and 205B can differ in slope angles of their backside 221b. In an embodiment, the backside 221b of the screw thread 205A can have a slope angle θ₂ of 90°. In an embodiment, the backside 221b of the screw thread 205B can have a slope angle θ₂ of less than 90°.

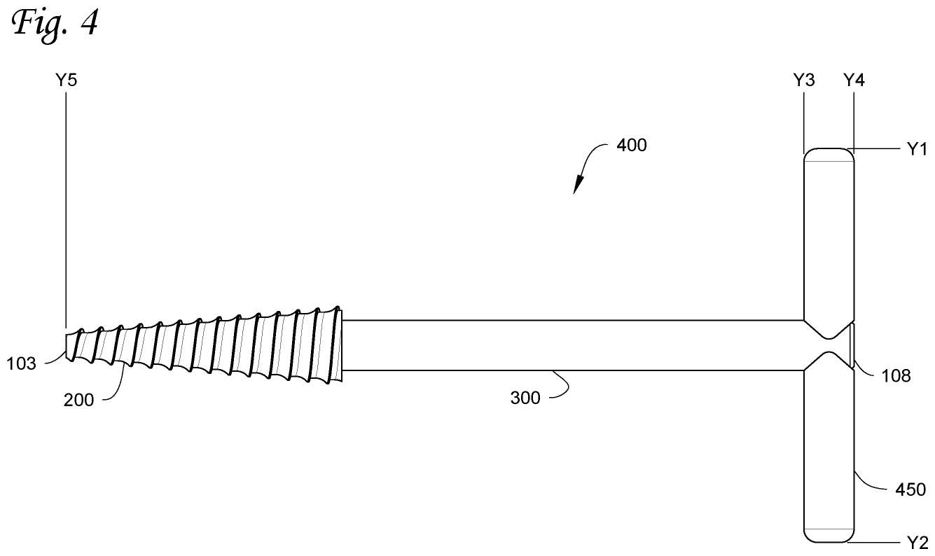

Referring to FIG. 4, FIG. 4 schematically illustrates an opening wedge tap instrument 400 according to another embodiment. This embodiment can include the opening wedge tap instrument 100's features discussed above. For simplicity of this specification, features that are similar or identical to those discussed above are not described in further detail. The opening wedge tap instrument 400 can include a handle 450, a screw 200, and a shank 300. The opening wedge tap instrument 400 is also cannulated. That is, the opening wedge tap instrument 400 contains a continuous through-hole extending from the end 103 to the end 108, e.g., for accommodating a guiding rod or wire.

In an embodiment, the opening wedge tap instrument 400 has a “T” configuration. In an embodiment, the opening wedge tap instrument 400 can have a configuration other than the “T” configuration, for example, a screwdriver configuration.

The handle 450 can adopt a structure and shape useful for rotating the screw 200. In an embodiment, the handle 450 can have an elongated body. In an embodiment, the handle 450 includes a knob. In an embodiment, the handle 450 is a knob. In an embodiment, the handle 450 is a screwdriver handle. In an embodiment, the handle 450 has a regular shape. In an embodiment, the handle 450 has an irregular shape. In an embodiment, the handle 450 has a rod configuration including but not limited to, for example, a cylindrical rod, triangular rod, square rod, rectangular rod, pentagonal rod, hexagonal rod, or octagonal rod, or the like, or a combination thereof. In an embodiment, the handle 450 is a straight rod. In an embodiment, the handle 450 is a curved rod.

The handle 450 is capable of being removably or irremovably attached to the shank 300. In an embodiment, the shank 300 can fit and secure into a hole of the handle that facilitates driving the screw 200 into an osteotomy cut through a screw-in motion. In an embodiment, the end 108 of the shank 300 can include a triangular, square, pentagonal, hexagonal, octagonal structure, or the like, or a combination thereof, so that the shank 300 can be secured into a handle. In an embodiment, the shank 300 removably attaches to the handle 450 via its end 108. In an embodiment, the shank portion 101a and the handle 200 are unitary one-piece construction, e.g., being machined from a single piece of material.

The handle 450 can have a length, which is indicated by the distance between lines Y1 and Y2, in a range from at or about 20 mm to at or about 200 mm, according to an embodiment. In an embodiment, the handle 450 has a length of no more than 30 mm, 40 mm, 50 mm, 60 mm, 70 mm, or 80 mm. In an embodiment, the handle 200 can have a length of at or about 50 mm. In an embodiment, the handle 200 can have a diameter or thickness, which is indicated by the distance between lines Y3 and Y4, in a range of at or about 5 mm to at or about 20 mm. In an embodiment, the handle 405 can have a diameter or thickness of at or about 8 mm. In an embodiment, the handle 405 can have a diameter or thickness of at or about 6 mm. In an embodiment, the handle 405 can have a diameter or thickness of at or about 4 mm.

In an embodiment, the opening wedge tap instrument 400 can have a length indicated by the distance between lines Y4 and Y5 in a range of at or about 80 mm to at or about 120 mm. In an embodiment, the length is at or about 100 mm.

Each of the screw 200, shank 300, and handle 450 can independently be made from a material with suitable strength, hardness, and/or rigidity. In an embodiment, the material includes but not be limited to, for example, surgical-grade metal alloys, stainless steel, chrome-molybdenum vanadium steel, carbon steel, titanium alloy, or the like, or a combination thereof. It will be appreciated that the material can also include but not be limited to, for example, porcelain, glass, composite material, or the like, or a combination thereof. In an embodiment, the material is an autoclavable material.

In an embodiment, the screw 200 is made of a biocomposite or bioceramic material that helps the bone grow while providing strength to hold the opening. In an embodiment, the material can include one or more selected from the group consisting of hydroxyapatite (HAP), Ca₁₀(PO₄)₆(OH)₂, tricalcium phosphate β(β TCP), and Ca₃(PO₄)₂.

The disclosure also provides a method for performing an osteotomy using the opening wedge tap instrument disclosed herein. In an embodiment, the method includes making an appropriate osteotomy cut on a selected position of the tibia.

In an embodiment, the method also includes drilling a pilot hole into the cut. In an embodiment, the method includes installing a guiding rod or wire, for example, k-wire, into the pilot hole. The pilot hole can be created by a suitable surgical drilling tool. In an embodiment, the surgical drilling tool drills into the tibia at a suitable depth to create the pilot hole. In an embodiment, the depth is at or about 2 to at or about 20 mm. In an embodiment, the depth is not more than at or about 10 mm.

In an embodiment, the method includes inserting the guiding rod or wire into the through-hole of the opening wedge tap instrument 100 or 400. In an embodiment, the method also includes installing the opening wedge tap instrument 100 or 400 onto the guiding rod or wire through its through hole 110. The guiding rod or wire can help to position the opening wedge tap instrument 100 or 400 at a suitable angle. In an embodiment, the suitable angle is 90°, thereby ensuring the screw 200 of the opening wedge tap instrument 100 or 400 be perpendicularly screwed into the osteotomy cut.

In an embodiment, the method includes rotating the opening wedge tap instrument 100 or 400 so that the screw 200 pries open the osteotomy cut to the pre-determined degree of correction through its screw-in motion.

In an embodiment, the method includes unscrewing the opening wedge tap instrument 100 or 400 so that its screw 200 moves out of the tibia. In an embodiment, the method further includes filling the hole left by the screw 200 with an implant. In an embodiment, the method includes fixating the osteotomy cut with a fixation plate.

In another embodiment, the method includes detaching the shank 300 from the screw 200 and then securing the osteotomy cut using a fixation plate. In an embodiment, the method also includes re-attaching the shank 300 to the screw 200 and unscrewing the screw 200 out of the tibia. In an embodiment, the method further includes filling the hole left by the screw with an implant.

In still another embodiment, the method includes fixating the desired degree of correction by securing the osteotomy cut with a fixation plate, and then unscrewing the opening wedge tap instrument 100 or 400 so that its screw 200 moves out of the tibia. In an embodiment, the method further includes filling the hole left by the screw with an implant.

In an embodiment, the implant can have a shape and size identical or substantially similar to those of the screw 200 of the opening wedge tap instrument 100 or 400. In an embodiment, the implant has a profile identical or substantially similar to that of the screw 200 of the opening wedge tap instrument 100 or 400. Thereby, the implant can provide an anatomical fit to the hole left by the screw 200 of the opening wedge tap instrument 100 or 400 when it is screwed or filled into the hole. The implant can be made of a biocompatible material. In an embodiment, the biocompatible material is compatible with the bone tissue of the tibia. In an embodiment, the biocompatible material is a bioceramic and/or biocomposite material. In an embodiment, the biocompatible material can include one or more selected from the group consisting of hydroxyapatite (HAP), Ca₁₀(PO₄)₆(OH)₂, tricalcium phosphate β(β TCP), and Ca₃ (PO₄)₂.

### ASPECTS

It is appreciated that any of aspects 1-16 can be combinable to any of aspects 17-19.

Aspect 1. An opening wedge tap instrument for osteotomy, comprising an elongated body including a shank and a screw,
wherein the screw is configured to a frustoconical shape.
the screw includes a thread including a flank, crest, and root,
the flank includes a driving side and backside,
the driving side is not symmetrically related to the backside,
the driving side has a smaller gradient relative to the longitudinal axis of the screw than the backside,
the crest includes a flat surface, and
the opening wedge tap instrument has a through-hole extending along the longitudinal axis of the opening wedge tap instrument for receiving a guidewire.

Aspect 2. The opening wedge tap instrument as in aspect 1, the driving side including two edges having an angle in a range of larger than 120° to less than 180°.

Aspect 3. The opening wedge tap instrument as in any one of aspects 1-2, wherein the crest includes a flat edge or surface.

Aspect 4. The opening wedge tap instrument as in any one of aspects 1-3, wherein the root includes a flat edge or surface.

Aspect 5. The opening wedge tap instrument as in any one of aspects 1-4, wherein the thread has a depth of at or about 0.5 mm.

Aspect 6. The opening wedge tap instrument as in any one of aspects 1-5, wherein the thread has a pitch of at or about 2.5 mm.

Aspect 7. The opening wedge tap instrument as in any one of aspects 1-6, wherein the screw has a cone angle in a range of at or about 8° to at or about 10°.

Aspect 8. The opening wedge tap instrument as in any one of aspects 1-7, wherein the screw has a minimum diameter of at or about 3.91 mm and a maximum diameter of at or about 10.03 mm.

Aspect 9. The opening wedge tap instrument as in any one of aspects 1-8, wherein the screw has a length at or about 35 mm.

Aspect 10. The opening wedge tap instrument as in any one of aspects 1-9, wherein the screw is made of biocomposite or bioceramic material capable of promoting bone growth.

Aspect 11. The opening wedge tap instrument as in any one of aspects 1-10, wherein the shank is removably attached to the screw.

Aspect 12. The opening wedge tap instrument as in any one of aspects 1-11, wherein the shank is irremovably attached to the screw.

Aspect 13. The opening wedge tap instrument as in any one of aspects 1-12, wherein the shank has a diameter or thickness of at or about 4 mm to at or about 8 mm.

Aspect 14. The opening wedge tap instrument as in any one of aspects 1-13, wherein the shank has a length of at or about 65 mm.

Aspect 15. The opening wedge tap instrument as in any one of aspects 1-14, further comprising a handle.

Aspect 16. The opening wedge tap instrument as in any one of aspects 1-15, wherein the elongated body is irremovably or removably attached to the middle of the handle, and the handle includes an elongated body or knob.

Aspect 17. A method of performing an osteotomy using the opening wedge tap instrument as in any one of aspects 1-16, comprising performing an osteotomy cut on a tibia;

mounting a guidewire into the osteotomy cut;

inserting the guidewire into the thorough hole of the opening wedge tap instrument; and screwing the opening wedge tap instrument into the osteotomy cut to pry open the osteotomy cut a degree for correcting the tibia deformity.

Aspect 18. The method of aspects 17, further comprising: fixating the pry opened osteotomy cut with a fixation plate and unscrewing the screw out of the tibia.

Aspect 19. The method of aspect 17, further comprising: unscrewing the screw so that the screw moves out of the tibia.

Aspect 20. The method of aspects 18-19, further comprising filling or screwing an implant into a hole left by the screw of the opening wedge tap instrument.

Aspect 21. The method of aspect 20, wherein the implant is made of a biocompatiable material.

Aspect 22. The method of aspect 21, wherein the implant is made of bioceramic or biocomposite material.

Aspect 23. The method aspects 20-22, wherein the implant is made of a biocompatible material comprising one or more selected from the group consisting of hydroxyapatite (HAP), Ca₁₀(PO₄)₆(OH)₂, tricalcium phosphate β(β TCP), and Ca₃ (PO₄)₂.

Aspects 24. The method of aspects 20-23, wherein the implant has a shape and size substantially similar or identical to those of the screw of the opening wedge tap instrument.

Aspect 25. The method of aspects 19-24, further comprising fixating the osteotomy cut with a fixation plate.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

We claim:

1. An opening wedge tap instrument for osteotomy, comprising an elongated body including a shank and a screw,
wherein the screw is configured to a frustoconical shape,
the screw includes a thread including a flank, crest, and root,
the flank includes a driving side and backside,
the driving side is not symmetrically related to the backside,
the driving side has a smaller gradient relative to the longitudinal axis of the screw than the backside,
the crest includes a flat surface, and
the opening wedge tap instrument has a through-hole extending along the longitudinal axis of the opening wedge tap instrument for receiving a guidewire.

2. The opening wedge tap instrument as in claim 1, the driving side including two edges having an angle in a range of larger than 120° to less than 180°.

3. The opening wedge tap instrument as in claim 1, wherein the crest includes a flat edge.

4. The opening wedge tap instrument as in claim 1, wherein the root includes a flat edge.

5. The opening wedge tap instrument as in claim 1, wherein the screw has a cone angle in a range of at or about 8° to at or about 10°.

6. The opening wedge tap instrument as in claim 1, wherein the thread has a depth of at or about 0.5 mm.

7. The opening wedge tap instrument as in claim 1, wherein the thread has a pitch of at or about 2.5 mm.

8. The opening wedge tap instrument as in claim 1, wherein the screw has a minimum diameter of at or about 3.91 mm and a maximum diameter of at or about 10.03 mm.

9. The opening wedge tap instrument as in claim 1, wherein the screw has a length of at or about 35 mm.

10. The opening wedge tap instrument as in claim 1, wherein the screw is made of biocomposite or bioceramic material capable of promoting bone growth.

11. The opening wedge tap instrument as in claim 1, wherein the shank is removably attached to the screw.

12. The opening wedge tap instrument as in claim 1, wherein the shank is irremovably attached to the screw.

13. The opening wedge tap instrument as in claim 1, wherein the shank has a diameter or thickness of at or about 4 mm to at or about 8 mm.

14. The opening wedge tap instrument as in claim 1, wherein the shank has a length of at or about 65 mm.

15. The opening wedge tap instrument as in claim 1, further comprising a handle.

16. The opening wedge tap instrument as in claim 1, wherein the elongated body is irremovably or removably attached to the middle of the handle, and the handle includes an elongated body or knob.

17. A method of performing an osteotomy using the opening wedge tap instrument as in claim 1, comprising
performing an osteotomy cut on a tibia;
mounting a guidewire into the osteotomy cut;
inserting the guidewire into the thorough hole of the opening wedge tap instrument; and
screwing the opening wedge tap instrument into the osteotomy cut to pry open the osteotomy cut a degree for correcting a tibia deformity.

18. The method of claim 17, further comprising: unscrewing the screw of the opening wedge tap instrument so that the screw moves out of the tibia.

19. The method of claim 18, further comprising: filling or screwing an implant into a hole left by the screw of the opening wedge tap instrument.

20. The method of claim 19, wherein the implant has a shape and size substantially similar or identical to those of the screw so that the implant anatomically fits the hole.
